(19) United States
(12) Patent Application Publication
(10) Pub. No.: US 2005/0059904 A1
(43) Pub. Date: Mar. 17, 2005

(54) Device and method for determining a hearing range

(76) Inventors: Josef Chalupper, Paunzhausen (DE); Thomas Hies, Erlangen (DE); Reinier Kortekaas, Erlangen (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 10/944,592

(22) Filed: Sep. 17, 2004

(30) Foreign Application Priority Data
Sep. 17, 2003 (DE) 10343007.5

(51) Int. Cl. A61B 5/00

(52) U.S. Cl. 600/559

(57) ABSTRACT

The object is to allow the adjustment of hearing aids without the use of sound-insulated acoustic booths. For this purpose provision is made for estimating a sound pressure level in front of the eardrum (5) from the sound pressure level in front of the hearing aid (2). Acoustic masking is determined from the estimated sound pressure level in front of the eardrum by means of a psycho-acoustic model. The actually measurable hearing range of the patient and therefore the optimum adjustment of the hearing aid can then in turn be determined from this.

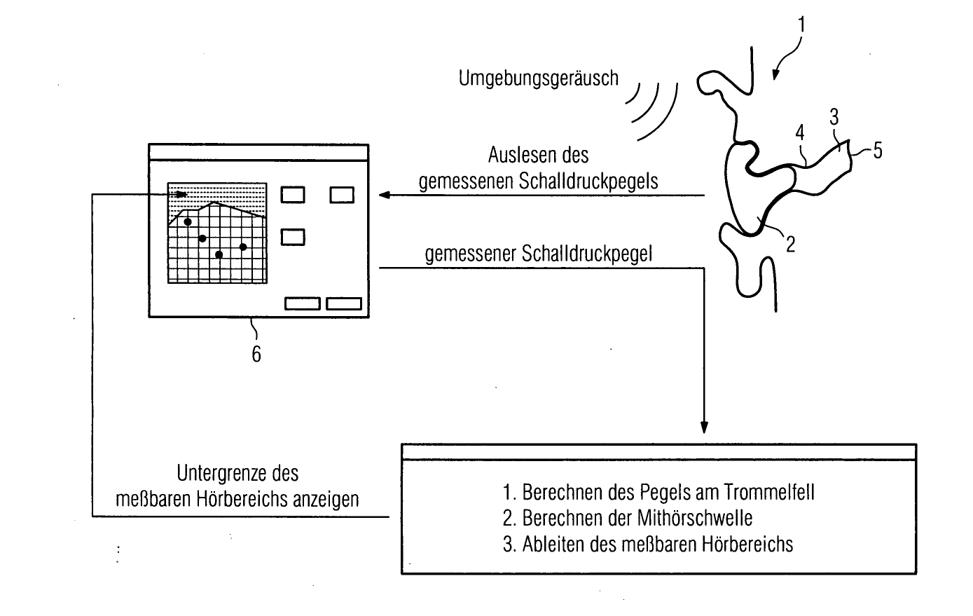

### CROSS REFERENCE TO RELATED APPLICATIONS

This application claims priority to the German application No. 10343007.5, filed Sep. 17, 2003 and which is incorporated by reference herein in its entirety.

### FIELD OF INVENTION

The present invention relates to a device for determining a hearing range and a corresponding method for this.

### BACKGROUND OF INVENTION

With so-called in-situ audiometry the hearing status of the individual hearing-impaired person is measured using sine tones or narrow-band noise signals, which are generated in the hearing aid. During such a measurement of hearing status a threshold measurement or loudness rating is carried out. In the case of the threshold measurement the hearing threshold and/or discomfort threshold is determined.

The advantage of in-situ audiometry is that all the factors which influence the acoustics in the auditory canal are in principle taken into account. The ear-piece of a behind-ear hearing aid has a particularly significant influence, as have the hearing aid insert of an in-ear hearing aid and the residual volume between the ear-piece or the hearing aid and the eardrum. With such in-situ audiometry the same device is used for measurement and treatment. It is hoped that this allows a better estimation of the sound pressure level at the eardrum and accordingly more precise adjustment of the amplification of the hearing aid to individual hearing loss.

Such methods for in-situ audiometry have been available for several years in the adjustment software of various hearing aid manufacturers, such as Widex, Phonak, Starkey, to name but a few. Completely mobile adjustment, including audiometry, is therefore possible in conjunction with a portable computer. One possible application would for example be adjustment in a retirement home.

### SUMMARY OF INVENTION

One problem that arises when determining hearing status however is that loud ambient noise can mask the quiet tones generated by the hearing aid during a hearing threshold measurement. This can be the case in particular with mobile adjustment, which is typically carried out without the use of a sound-insulated acoustic booth. In a loud environment the patient can only discern loud, i.e. more highly amplified, tones, which are no longer masked. As a result the hearing loss can be classed as greater than it actually is. The amplification of the hearing aid would therefore be set at a higher level than is necessary or desirable. There is also the problem that in natural surroundings the level of the acoustic environment varies significantly from room to room and generally also varies significantly in one room over time. It is therefore not possible to determine the degree of masking by ambient noise beforehand.

In this context it is however known from the devices from the Starkey company that the inherent noise generated by the hearing aid itself is taken into account in different methods. This inherent noise is produced for example in the final stage by quantization effects. In a sound-insulated room this inherent noise determines the lower limit of the theoretically measurable hearing threshold. This lower limit can be displayed in the software interface of the adjustment software.

An object of the present invention is therefore to ensure better adjustment of hearing aids even in rooms without sound insulation.

According to the invention this object is achieved the claims. The hearing range signifies the objective range, within which a subjective hearing threshold can be measured. In other words the lower limit of the measurable hearing range represents the lowest measurable hearing threshold.

The invention also provides a method for determining a hearing range by measuring the sound pressure level of ambient noise, estimating a sound pressure level in front of the eardrum of a patient wearing a hearing aid, at least as a function of the design of the hearing aid or earpiece and the measured sound pressure level of the ambient noise and determining a measurable hearing range based on the estimated sound pressure level in front of the eardrum.

One advantage of the inventive solution is that no additional equipment, e.g. a sound level meter, is required to determine whether a certain room is suitable for taking audiometric measurements. Also the computer can convert free-field sound pressure levels to corresponding masking thresholds. The lower limit of the measurable hearing range can also be monitored continuously during measurement.

In a preferred embodiment the measuring device is integrated into the hearing aid. The sound pressure level can thereby be determined immediately in front of the hearing aid in a suitable manner without additional resources.

The sound pressure level of the ambient noise can be measured in a broad band or frequency-specific manner. Frequency-specific measurement has the advantage that different masks can be taken into account in a differentiated manner.

The estimator and the evaluator can be integrated into an adjustment device for the acoustic adjustment of a hearing aid to a patient. This adjustment device generally comprises a PC, which is connected to the hearing aid in a wireless or non-wireless manner. The required adjustment software is installed on this PC, which is typically operated by an acoustician.

In the estimator it is possible to take into account the inherent noise of the hearing aid when estimating the sound pressure level in front of the eardrum. Taking into account inherent noise in this way improves the estimation of the sound pressure level in front of the eardrum, as this is not only caused by ambient noise.

The lowest measurable hearing threshold is preferably determined in the evaluator by means of a psycho-acoustic model. This allows a mask spectrum and therefore a corresponding hearing threshold to be estimated precisely based on the ambient noise spectrum and the inherent noise spectrum.

### BRIEF DESCRIPTION OF THE DRAWINGS

The present invention is described below in more detail with reference to the accompanying drawing. The accompanying figure depicts an outline of the signal processes for determining a hearing range.

### DETAILED DESCRIPTION OF INVENTION

The exemplary embodiment described below represents a preferred embodiment of the present invention.

A patient 1 wears a hearing aid 2. A residual volume 3 in the auditory canal 4 of the patient 1 is defined on the one hand by the eardrum 5 and on the other hand by the hearing aid 2. This residual volume 3 determines the sound pressure level in front of the eardrum 5 to a significant degree.

The hearing aid 2 measures the sound pressure level of ambient noise. The measured sound pressure level is read from the hearing aid 2 by a PC 6, on which adjustment software is installed. There is a wireless or non-wireless communication connection between the PC 6 and the hearing aid 2 for this purpose.

The adjustment software 7 calculates the level at the eardrum 5 from the sound pressure measured. From this it calculates the masking threshold resulting from the masking caused by the sound pressure level at the eardrum. The hearing range actually available is finally derived from the masking threshold. The lower limit of the established hearing range is displayed graphically on the PC 6. In the example the hearing range is below the continuo us curve. This is obtained from the masking threshold corrected by the masking caused by ambient noise. This means that the acoustician can determine the hearing range of the patient regardless of ambient noise. The acoustician can use the lower limit of the measurable hearing range to establish whether a room is suitable for determining hearing thresholds. The acoustician can also establish whether a measured hearing threshold is an actual threshold or a threshold resulting from masking. It is then possible to adjust the hearing aid in an optimum manner, in particular the amplifications in the individual frequency bands.

1-11. (cancelled)

12. A device for determining a hearing range of a patient, comprising:
a measuring device for measuring a first sound level of an ambient noise;
a first processing device for determining a second sound level in front of an eardrum relative to the direction of sound aiming toward the eardrum of the patient wearing a hearing aid, using at least one design parameter of the hearing aid or of an earpiece thereof and the measured first sound level; and
a second processing device for determining the hearing range using the second sound level.

13. The device according to claim 12, wherein the design parameter is chosen from the group consisting of volume, diameter, length, width, depth, height, shape, type of material, density and weight.

14. The device according to claim 12, wherein the measuring device is integrated into the hearing aid.

15. The device according to claim 12, wherein the measuring device is adapted to measure the first sound level related to at least one selected frequency within the frequency spectrum of the ambient noise.

16. The device according to claim 12, wherein the first and the second processing units are integrated into an adjustment device for adjusting the hearing aid to acoustic needs of the patient.

17. The device according to claim 12, wherein the first processing device uses an inherent noise of the hearing aid to determine the second sound level.

18. The device according to claim 12, wherein the second processing device uses a psycho-acoustic model of a masking related to sound processed by the hearing aid drowned by loud ambient noise for determining the hearing range.

19. A method for determining a hearing range, comprising:
measuring a first sound level of an ambient noise;
calculating a second sound level in front of an eardrum of a patient wearing a hearing aid, using at least one design parameter of the hearing aid or of an earpiece thereof and the measured first sound level; and
determining the hearing range using the second sound level.

20. The method according to claim 19, wherein the first sound level is measured in the hearing aid and transmitted wirelessly to an adjust ment device used to calculate the second sound level and to determine a hearing threshold level of the hearing range.

21. The method according to claim 19, wherein the first sound level is measured related to at least one selected frequency within the frequency spectrum of the ambient noise.

22. The method according to claim 19, wherein calculating the second sound level further uses an inherent noise of the hearing aid.

23. The method according to claim 19, wherein determining the hearing range further uses a psycho-acoustic model of a masking related to sound processed by the hearing aid drowned by loud ambient noise.
